(19) United States
(12) Patent Application Publication
(10) Pub. No.: US 2004/0019080 A1
(43) Pub. Date: Jan. 29, 2004

(54) Prevention and treatment of migraine and other recurrent headaches using leukotriene LTD4 receptor blocker drugs

(76) Inventors: Fred D. Sheftell, Stamford, CT (US); Robert C. Kevorkian, West Granby, CT (US)

Correspondence Address: Patrick D. Kelly, 11939 Manchester #403, St. Louis, MO, 63131, US

(21) Appl. No.: 09/794,457

(22) Filed: Feb. 27, 2001

Related U.S. Application Data
(63) Continuation-in-part of application No. 09/221,015, filed on Dec. 23, 1998, now Pat. No. 6,194,432.
(60) Provisional application No. 60/103,933, filed on Oct. 13, 1998.

(51) Int. Cl. A61K 31/47

(52) U.S. Cl. 514/311

(57) ABSTRACT

Receptor blocker (antagonist) drugs which suppress the binding of the D4 class of leukotriene molecules to leukotriene receptors can reduce the frequency and severity of recurrent primary headaches, including migraine and cluster headaches. Various LTD4 receptor blockers are commercially available, including montelukast and zafirlukast. In the past, these drugs have been used to treat asthma. During human clinical trials on asthma patients, headaches were one of the most commonly reported side effects of those two drugs; therefore, they apparently were never tested as agents which might prevent or reduce migraine or cluster headaches. However, it has been shown that in at least some patients who suffer from acute recurrent migraines, LTD4 receptor blockers can greatly reduce the frequency, duration, and/or severity of their headaches, when taken chronically on a daily basis. Tests also indicate that LTD4 receptor blockers can be combined with a triptan drug to achieve a synergistic effect against acute headaches.

### RELATED APPLICATION

This application is a continuation-in-part of application Ser. No. 09/221,015, filed Dec. 23, 1998, issued as U.S. Pat. No. 6,194,432 on Feb. 27, 2001. That application claimed priority based on provisional application 60/103,933, filed Oct. 13, 1998.

### BACKGROUND OF THE INVENTION

This invention is in the field of pharmacology, and relates to drugs that can help reduce the frequency, duration, and/or severity of certain types of headaches classified as “recurrent primary headaches” (including migraine headaches and cluster headaches). This treatment involves daily or other chronic administration of certain types of “leukotriene antagonist” drugs which can suppress activation of the “D4” subtype of receptor that is normally activated by leukotrienes. Several such leukotriene D4-receptor antagonist drugs are known; previously, they have been used for treating asthma, as discussed below.

Migraine headaches (also referred to simply as migraines, for convenience) and cluster headaches are discussed in numerous journal articles and in various full-length medical texts, such as Headache in Clinical Practice (edited by S. Silberstein et al., Oxford Univ. Press, 1998); Headache Classification and Epidemiology by J. Olesen (Raven Press 1994), and Headache Disorders: A Management Guide for Practitioners, by A. Rapoport and F. Sheftell (W. B. Saunders, Philadelphia, 1996). In addition, various definitions, categories, and diagnostic standards have been approved and issued by the International Headache Society (IHS), and were published as a supplement to the journal Cephalalgia in 1988.

Migraines and cluster headaches are important, well-known, and extensively studied medical problems, and they can be regarded as “recurrent primary headaches”. They are recurrent, since they recur with sufficient frequency to seriously interfere with the health and quality of life of a patient, to a point of requiring and demanding medical attention (as opposed to just taking an over-the-counter analgesic and lying down till the pain decreases). They are “primary” headaches since they usually arise as a primary condition, independently of other causative conditions such as tumors, sinus or other infections, bleeding problems, etc.

A third major category of recurrent primary headaches is often referred to as “tension” (or “tension-type”) headaches. Although these can often be resolved in many patients if the source of the tension can somehow be eliminated or substantially lessened, that approach may require a major lifestyle change for the patient, and is often impractical or impossible for patients who cannot escape from the demands imposed by stressful work, family, or other situations. Accordingly, recurrent tension headaches must often be treated as a medical problem using drug intervention, usually in combination with training in relaxation and stress management techniques. In addition, many researchers and physicians believe that tension headaches and migraine headaches exist on a continuum, and involve the same or overlapping neurobiological mechanisms. It should also be noted that various drugs (including anti-inflammatory drugs, such as certain types of prostaglandin antagonists) that are effective (in at least some patients) in treating migraine headaches are also effective in treating tension headaches as well. Because of their similarities and overlapping factors, it is believed by the Applicant that tension headaches may be susceptible to effective treatment, in at least some sufferers, using LTD4 receptor blocker drugs as disclosed herein.

There are at least three “aspects” or “traits” of primary recurrent headaches that are important in this invention, since these traits can provide quantifiable evidence of whether a treatment is or is not effective in controlling headaches, either in a specific patient, or across a population of patients large enough to generate useful statistics. Those three aspects are: (1) frequency, which is usually evaluated over a span of time, such as number of such headaches per week, per month, or per year; (2) duration, which evaluates (usually in hours) how long a headache lasts, from the time it begins to develop into a migraine or cluster headache, until it has been resolved; and, (3) severity (also referred to as intensity), which is based on subjective estimates of the severity or intensity of pain or other side effects (such as nausea) being suffered by patients during such headaches.

If a preventive drug treatment can significantly reduce any of these three aspects, it can be regarded as effective and beneficial, since it can substantially improve the quality of life for such patients. For obvious reasons, an ideal A preventive treatment would reduce all three aspects; and, indeed, the preventive treatment disclosed herein does indeed appear to accomplish that ideal goal, in at least some patients, based on both: (i) an initial open-label trial, and (ii) follow-up evidence that has accumulated during the past two years.

However, it must be recognized that simultaneously reducing all three aspects (frequency, duration, and severity) of recurrent primary headaches is not essential to providing useful, effective, beneficial relief from severe headaches. A treatment which can reduce any one (or two) of those traits is effective and useful from a medical perspective, and will be enthusiastically welcomed by sufferers of migraine and/or cluster headaches (and by their families and friends).

Migraines are more common than cluster headaches, and have been studied more extensively. In addition, a better and more effective set of drugs have been developed to treat migraines, than cluster headaches. For those reasons, the discussion below focuses mainly on migraines, rather than cluster headaches. However, because of various physiological and pharmacological factors, and because of the highly positive results observed so far in initial tests on migraine sufferers, it is believed by the Applicant that chronic treatment with leukotriene D4 (LT-D4) receptor blocker drugs, as disclosed herein, is also likely to provide significant benefits to at least some patients who suffer from cluster headaches, or from other types of recurrent primary headaches that do not respond adequately to other previously-known treatments.

It should also be recognized that migraines are often “triggered” or aggravated by certain factors, which vary widely among different patients. In some patients, migraines are triggered by eating certain foods, such as chocolate, red wine, MSG, artificial sweeteners, or certain types of cheese. In some patients, migraines can be triggered by perfumes or other compounds that generate odors. In women, migraines often accompany menstruation.

Accordingly, if a preventive drug treatment can reduce the susceptibility of a patient to severe headaches by one or more triggering factors, that drug treatment is effective and useful. However, susceptibility factors are regarded herein as being fully included within the three factors listed above, since a treatment which reduces a patient's susceptibility to one or more triggering factors will reduce the frequency, duration, and/or severity of severe headaches suffered by that patient.

Lack of Effective Preventive Treatments

Drug treatments for migraine headaches have improved substantially during the past few years, with the widespread introduction and use of drugs known as “triptans”. These include sumatriptan (sold under tradenames such as IMITREX and IMIGRAN by Glaxo-Wellcome, and also used to treat cluster headaches), naratriptan (sold under the tradenames AMERGE and NARAMIG, also by Glaxo-Wellcome), zolmitriptan (sold under the name ZOMIG, by Zeneca Pharmaceuticals), and rizatriptan (sold under the name MAXALT, by Merck). All of these are available as tablets for oral ingestion; in addition, for patients who suffer from nausea, several of these drugs are also available in other forms, such as subcutaneous injectable formulations, nasal sprays, and wafers or troches designed to dissolve in the mouth.

The primary mode of action of all of these “triptan” drugs is believed to involve selective activation of certain serotonin receptors subtypes, primarily 5HT-1B receptors, which are present on blood vessels, and 5HT-1D receptors, which are present on nerve cell terminals, both peripherally and in the central nervous system (CNS).

Serotonin is the common name for 5-hydroxytryptan, abbreviated as 5-HT. Inside brain and spinal cord tissue, 5-HT is a neurotransmitter that is generally inhibitory, since it suppresses (rather than activates) nerve signals in neurons. In vascular tissue, sumatriptan and other “triptan” drugs generally cause constriction of blood vessels in the cerebral region, and help to reverse neurogenic inflammation around those blood vessels during migraine attacks.

In general, triptan drugs (and other therapies that are suited for treatment of acute migraine headaches that have already emerged) cannot be used as chronic preventive treatments, for a number of reasons. One of the main reasons is that chronic administration of acute-care drugs (such as triptan drugs) to migraine patients often drives a patient into a state where the patient suffers “rebound” headaches and/or chronic headaches. Indeed, chronic administration of triptan drugs has been observed to generate “transformed” migraine headaches which occur daily. This is discussed in sources such as Neurologic Clinics: Advances in Headache (N. T. Mathew, editor; W B Saunders, Philadelphia, Pa., 1997).

Triptan drugs also pose a risk of adverse cardiovascular events, such as heart attack, stroke, etc., so they are contraindicated in patients suffering from heart disease, stroke, uncontrolled hypertension, basilar or hemiplegic migraine, and in people taking various other drugs, such as monoamine oxidase inhibitors. Accordingly, before triptan drugs can be prescribed safely, the diagnosing physician must do a risk factor analysis for various potential cardiovascular disorders, especially among patients who may be overweight or who smoke, or who suffer from high cholesterol, inadequate exercise levels, hypertension, a family history of heart disease or stroke, etc. Clearly, the risks involved in any such analysis would increase substantially if a doctor or patient were tempted to use triptan drugs as a chronic treatment.

Regrettably, standard preventive pharmacologic treatments (involving, for example, aspirin, acetaminophen, ibuprofen, ergotamine or its derivatives, etc.) simply does not work in the large majority of migraine patients. Migraine patients who take such drugs on a daily basis usually observe no significant reduction in the frequency, duration, or severity of their headaches. In addition, most of these drugs suffer from toxicity syndromes, when used at the dosage rates that migraine and cluster sufferers are tempted to use, for preventive purposes. Ulcers and other gastrointestinal bleeding and “8th nerve” toxicity often result when aspirin is used chronically and at unusually high dosages; hepatotoxicity can result from acetaminophen overuse; and various types of kidney damage can arise from overuse of these and other types of over-the-counter analgesics (it has been estimated that 10% of all end-stage kidney disease is secondary to the overuse of over-the-counter non-steroidal medications). Addiction also may occur with prescription pain-killers such as butalbital products (Fiorinal, Fioricet, Esgic, etc.) or opiates (Percocet, Percodan, Codeine, Stadol, etc.).

In addition to those problems, prior efforts at chronic preventive treatments for migraine or cluster headaches also frequently resulted in undesired side effects such as fatigue, decreased energy, depression, weight gain, decreased libido, dry mouth, cardiac arrhythmias, hair loss, tremors, hepatotoxicity, etc. Most such chronic preventive efforts do not maintain their efficacy over sustained periods of time, and a number of such therapies require periodic monitoring of blood (including complete blood count (CBC), platelet counts, blood urea nitrogen levels, creatinine, and cholesterol levels), as well as tests to ensure that liver and/or kidney functioning has not been impaired.

In view of the important advances and options offered by the recent development of triptan drugs, it is widely agreed among headache specialists that preventive therapies have not kept pace with advances in acute therapy. All of the previously known preventive strategies (with the possible exception of very recent discoveries using injections of diluted botulin toxin) are associated with potentially serious limitations, adverse events, and side effects, all of which makes their use unattractive to doctors and patients. Even when a preventive therapy is deemed to be suitable for testing in a specific patient, the results usually show, at best, only about a 50% decrease in frequency and intensity, in about half of the patients tested on such treatment regimens.

In general, the best preventive approach that treating physicians can take under the prior art involves efforts to control any potential triggering factors (such as careful screening of the patient's diet and environment to identify triggering factors, so the patient can take extra precautions to avoid them), and treatment of any concomitant medical problems that may help trigger migraines. For example, anti-depressants are often prescribed for patients whose migraines appear to be triggered or aggravated by depression, and beta-blockers (which help regulate heartbeat rates) are prescribed for patients whose migraines appear to be triggered by fluctuations in blood pressure or heartbeat. Beta-blockers are the most commonly prescribed treatment that might be regarded as a preventive treatment for migraine. However, beta-blockers are contraindicated in patients with asthma, and there is a high correlation between asthma and migraines; an estimated 20 to 30 percent of asthmatic patients suffer from migraines.

For various similar reasons, there also are no truly effective strategies for preventing cluster headaches.

In summary, there is a severe and very serious lack of effective and adequate preventive treatments to reduce the frequency, duration, and/or severity of migraine or cluster headaches. Accordingly, there is a major medical need for effective preventive drug treatments that can be used in a chronic and long-term manner to prevent migraine or cluster headaches (rather than just for treating them such headaches, once they have commenced), and to reduce their duration and severity when they do arise.

In addition, there is also an important medical need for improved drug treatments that can decrease the amount of pain and suffering caused by migraine or cluster headaches, once they begin. One such form of treatment would involve administration of a drug that can help reduce migraine symptoms in patients who are not adequately helped by the triptan class of drugs. Another such treatment would involve coadministration of two completely different types of drugs, which would work by completely different and independent mechanisms, to provide better pain relief than either class of drug can provide by itself.

Background Information on Leukotrienes

Since this invention involves certain types of drugs that act as “leukotriene antagonists”, this section provides background information on leukotrienes, and on drugs used to reduce leukotriene activity in asthma patients.

Leukotrienes are naturally-occurring molecules that function in mammals as inter-cellular messengers (i.e., hormones). A good brief overview of their scientific history is provided in a “Research News” article in Science (Marx 1982; complete citations to articles are provided below). A full-length book on leukotrienes is also available (J. Rokach, ed., Leukotrienes and Lipoxygenases (Elsevier Publ., Amsterdam, 1989)).

Very briefly, in 1938, an Australian named Charles Kellaway discovered “slow reacting substances” (abbreviated as SRS), which could cause slow contractions of smooth muscles. For the next 40 years, numerous researchers studied these SRS compounds, and divided them into different categories. One set of these compounds were labelled “SRS-anaphylaxis” (SRS-A), because they were released during or after allergic responses. Even before SRS-A had been isolated, identified, or properly named, “investigators were interested in SRS because they thought it might help trigger asthma attacks by contracting the smooth muscle of the respiratory tract and causing airway obstruction” (quoted from Marx 1982).

“SRS” managed to defy all efforts at chemical analysis for 40 years after its initial discovery. Finally, in 1979, scientists realized it was a mixture of at least three different but similar molecules, all of which are large and have complex structures. Soon after that, yet another version was discovered, with an unstable epoxide ring; it was soon realized that the epoxide version was a chemical precursor to the other more stable leukotriene molecules.

These compounds were already the subject of intense interest before their molecular structures were known, because of their extraordinary potency as bronchoconstrictors. Therefore, as soon as their chemical structures were finally known, numerous drug companies promptly began having their scientists search for drugs that could bind to leukotriene receptors without triggering the same physiological responses.

Their principle approach, which was quite logical and well-reasoned, involved making “chimera” molecules, with a portion of a leukotriene molecule bonded to some other type of molecule. The goal of this effort was to identify chimeric partially-leukotriene derivatives that would bind to LT receptors (acting through the leukotriene portion of the chimeric molecule), but which either: (i) would not trigger the same cellular reactions that natural LT molecules trigger, due to steric hindrance or other effects causes by the non-LT portion of the chimera; or, (ii) would stay bonded to an LT receptor for an abnormally long period of time, in the hope that a receptor would react only once with the drug molecule, and then would remain occupied by that drug for a long period of time, making it unavailable to react with natural LT molecules.

That approach worked quite well. Both montelukast (sold by Merck under the trademark SINGULAIR) and zafirlukast (sold by Astra-Zeneca under the trademark ACCOLATE) are chimeric molecules, having an LT segment (which will react with LT receptors) bonded to a foreign segment (which somehow fouls the LT receptor react-and-release mechanism). An extraordinary number of U.S. patents have been issued on potential LT receptor-blocking drugs; however, it is often difficult or impossible to determine whether the compounds listed in a patent on LT antagonist compounds are receptor-blocking drugs, or biosynthesis inhibitor drugs. Indeed, one should assume that the chemists and attorneys who drafted those patents typically did not know which type of activity(ies) any particular compound would have, as those patents were being drafted.

A database search identified at least 100 such patents, and most such patents appear to disclose and claim at least 20, often more than 60, and occasionally more than 150 distinct chemical structures. U.S. Pat. No. 5,565,473 (Belley et al 1996; assigned to Merck Frosst Canada) offers a good example; it lists at least 165 distinct chemical structures, and it describes the steps for synthesizing a large number of those compounds (one of which became montelukast, the commercial product). However, it makes no apparent effort to predict which of those 165 compounds would be leukotriene receptor blockers, and which would be leukotriene biosynthesis inhibitors.

Another example of a patent that lists a large number of leukotriene antagonists is U.S. Pat. No. 4,859,692 (Bernstein et al 1989). It is noteworthy because one of the compounds it lists became zafirlukast, the commercial product.

Returning to the natural forms of leukotriene molecules in humans, there are now at least five known subtypes, referred to by the designations LTA4, LTB4, LTC4, LTD4, and LTE4. All of these subclasses of leukotriene molecules are formed from arachidonic acid, a molecule with 20 carbon atoms, with four internal double bonds near the center of the chain, and with a carboxylic acid group at one end.

Arachidonic acid is continuously synthesized at cell membranes, by cleavage of certain types of phospholipids. This cleavage reaction is catalyzed by phospholipase enzymes. Free arachidonic acid is then converted into any of four different types of compounds, which are leukotrienes, prostaglandins, prostacyclins, and thromboxanes. All four of these types of compounds are called “eicosanoids”.

Prostaglandins, prostacyclins, and thromboxanes all contain cyclic structures, and are created when “cyclooxygenase” enzymes (often abbreviated as COX enzymes) generate cyclic structures from the carbon atoms in arachidonic acid. Recently-commercialized “COX inhibitor” drugs such as celecoxib (sold by a Monsanto-Pfizer consortium under the trademark CELEBREX) and rofecoxib (sold by Merck under the trademark VIOXX) are of great interest, both for inhibiting arthritis pain that cannot be treated adequately by other drugs, and possibly for helping treat (or reduce the risk of) certain types of cancer, including colon cancer.

Unlike the other three classes of eicosanoids, leukotrienes do not involve cyclooxygenase enzymes; instead, leukotrienes are created from arachidonic acid by a completely different enzymatic pathway. In this pathway, the double bonds in arachidonic acid are rearranged to create a “conjugated triene” structure, with three double bonds that alternate with single bonds; it is this structure which gives leukotrienes their “tri-ene” name.

LTA4 has an epoxide structure which is relatively reactive and unstable; accordingly, LTA4 serves mainly as a precursor, during the synthesis of the other leukotrienes. LTB4 is generated when the epoxide form is been hydrolyzed into a di-hydroxy compound. LTC4, LTD4 and LTE4 are all modified by the addition of cysteine, an amino acid that contains a relatively reactive sulfhydryl group (—SH) at the end of a spacer chain; accordingly, these “cysteinyl leukotrienes” are often referred to as “cysLT” compounds. The structures of all of these leukotrienes are known, and are illustrated in numerous reference works, including the Merck Index.

All of the eicosanoid compounds (including leukotrienes) tend to aggravate inflammation, pain, and fever, and they have been the targets of extensive research on anti-inflammatory and analgesic drugs. For example, anti-inflammatory steroids such as cortisone function by suppressing the phospholipase enzymes that cleave off arachidonic acid from membrane phospholipids. Pain-killers such as aspirin and ibuprofen act by blocking (to some extent) the cyclooxygenase enzymes that control the conversion of arachidonic acid to prostaglandins, prostacyclins, and thromboxanes.

Leukotrienes have been recognized as inflammatory agents since the early 1980's. Articles from that period which focus on specific cellular activities of the various leukotrienes include Smith et al 1980, Ford-Hutchison et al 1980, Bray et al 1981, Lewis 1981, Gimbrone et al 1984, and Levine et al 1984. Review articles from that period include Bray 1983 and Piper 1984.

In particular, leukotrienes are extremely potent broncho-constrictors. They range from roughly 100 to 1000 times more potent than histamine in reducing the internal diameters of bronchial passageways. For this reason, leukotriene antagonists are highly useful in combatting asthma.

There are also several distinct types of leukotriene receptors, as described in articles such as Metters 1995 and Nicosia et al 1999. Cross-affinities are known to exist between various leukotrienes and leukotriene receptors, as well as between leukotriene antagonist drugs and leukotriene receptors. For example, Aharony 1998 reports that both zafirlukast and montelukast appear to block LTD4 and LTE4 receptors, but do not appear to block LTC4 receptors.

Leukotriene Biosynthesis Inhibitors

A substantial number of US patents were issued in the 1980's and early 1990's, claiming that certain compounds known as “leukotriene biosynthesis inhibitors” could be used to prevent and/or treat migraine headaches. Such patents that were assigned to the Warner-Lambert company include U.S. Pat. No. 4,602,023 (Kiely et al 1986); U.S. Pat. No. 4,775,677 (Connor et al 1988); U.S. Pat. No. 4,786,755 (Kiely et al 1988); U.S. Pat. No. 4,810,716 (Connor et al 1989); U.S. Pat. No. 4,868,195 (Carethers et al, 1989); U.S. Pat. No. 4,868,199 (Carethers et al 1989); U.S. Pat. No. 4,868,200 (Carethers et al 1989); U.S. Pat. No. 4,868,205 (Carethers et al 1989); and U.S. Pat. No. 5,142,095 (Connor et al 1992).

U.S. patents assigned to Merck Frosst Canada on leukotriene biosynthesis inhibitors include U.S. Pat. No. 5,081,138 (Gillard et al 1992); U.S. Pat. No. 5,093,356 (Girard et al 1992); U.S. Pat. No. 5,102,881 (Zamboni et al 1992); U.S. Pat. No. 5,190,968 (Gillard et al 1993); U.S. Pat. No. 5,204,344 (Prasit et al 1993); U.S. Pat. No. 5,221,678 (Atkinson et al 1993); U.S. Pat. No. 5,225,421 (Gillard et al 1993); U.S. Pat. No. 5,232,916 (Zamboni et al 1993); U.S. Pat. No. 5,252,585 (Frenette et al 1993); U.S. Pat. No. 5,254,567 (Down et al 1993); U.S. Pat. No. 5,272,145 (Prasit et al, 1993); U.S. Pat. No. 5,273,980 (Frenette et al 1993); U.S. Pat. No. 5,290,798 (Gillard et al 1994); U.S. Pat. No. 5,308,850 (Gillard et al 1994); U.S. Pat. No. 5,334,719 (Frenette 1994); U.S. Pat. No. 5,380,850 (Prasit et al 1995); and U.S. Pat. No. 5,389,650 (Frenette et al 1995).

Still other drugs which reportedly can inhibit the synthesis of LT molecules include BAYx1005 (see Hamilton et al 1997 and Dahlen et al 1997), MK-886 (Friedman et al 1993), MK-0591 (Diamant et al 1995), ZD2138 (Nasser et al 1994), and zileuton, also known as A-64077 (Knapp 1990 and Hui et al 1991).

In addition, various published articles report physiological correlations between migraine headaches and leukotriene concentrations in the blood. These include Parantainen et al 1986, Gazzaniga et al 1987, Moskowitz 1990, and LaMancusa et al 1991.

Despite the large amount of work that went into those research and patenting efforts, none of those compounds (to the best of the Applicants' knowledge and belief) were ever approved, commercialized, or even tested for use in preventing or treating migraines, prior to the recent disclosure by one of the Applicants herein (Sheftell et al 1999) that leukotriene antagonists, taken chronically, can help prevent and reduce the intensity of migraines.

The reasons for the failure of numerous obviously interested companies to commercialize a potentially huge product is rarely published; accordingly, outside observers can only guess at what most likely happened, in light of known facts. In this particular case, two particular known facts are highly relevant, and worth attention. The first is this: if a leukotriene biosynthesis inhibitor is administered to a patient, the direct result will be to reduce the ability of the leukotriene-generating enzyme system to help handle the biochemical load of arachidonic acid that is being generated continuously inside any patient. As noted above, arachidonic acid is normally converted into four types of eicosanoids: prostaglandins, prostacyclins, thromboxanes, and leukotrienes. This means that the leukotriene-enzyme system normally handles and disposes of a substantial fraction of the arachidonic acid which is continuously being generated by cellular processes inside a patient. Therefore, one must assume that if the leukotriene biosynthesis system is suppressed by a leukotriene biosynthesis inhibitor drug, the quantity of arachidonic acid that normally passes through the leukotriene system will be forced to find other metabolic pathways for processing and eventual disposal. This will necessarily drive a patient's biochemical equilibrium toward the production of more prostaglandins, prostacyclins, and/or thromboxanes. That is a highly important fact, since each of those three classes of compounds can trigger potentially serious problems, if they begin to accumulate at excess levels.

The second highly relevant fact is this: the leukotriene biosynthesis inhibitor with the most extensive record of widespread public use is zileuton, which is sold as a drug to help control asthma, under the trademark ZYFLO, by Abbott Laboratories (North Chicago, Ill.). A search of the National Library of Medicine database “MEDLINE” indicated that not a single article has ever been published on the use of zileuton for treating or preventing headaches. It appears highly likely that one of the reasons zileuton was never even studied, to evaluate its potential for use in preventing migraine or other types of headaches, is that it actually caused headaches, rather than preventing them, in the clinical trials that were carried out on asthma patients. The Physician's Desk Reference (52nd edition, 1998; this same information is also reprinted in the “package inserts” that are sold with the drug) reports that when zileuton was taken 4 times daily at 600 mg, in various clinical trials, 24.6% of all users reported headaches, as an unwanted side effect.

That result needs to be put into perspective by pointing out that in the same clinical trials, almost exactly the same percentage (24.0%) of the control (placebo-treated) group from the tested population of asthma sufferers reported headaches as an unwanted side effect, when they took a harmless placebo drug.

Nevertheless, it seems fairly clear that the makers of zileuton did not bother testing it, as a potential preventive or treating agent for headaches, after large numbers of test subjects, in clinical trials, reported that they got headaches shortly after they took zileuton. It also must be noted that the frequency of reported headaches increased, in the treated test populations compared to the untreated control populations. Even though that increase was small, and not statistically significant, it nevertheless was indeed an increase. Both of those factors teach directly away from the current invention.

Accordingly, after the results of the asthma trials began to become available, until the first published reports of the invention by the Inventors herein, it appears that no pharmaceutical company or academic researcher working on or studying leukotriene biosynthesis inhibitors ever showed (or even suspected) that leukotriene biosynthesis inhibitors might offer an effective treatment for preventing migraine or cluster headaches.

However, soon after the first reports of this invention were published in mid-1999, research by others commenced on the possible use of leukotriene biosynthesis inhibitors as preventive treatments for migraines, and favorable results have now been published. The first known example is Grossman et al 2000 (published in November 2000), which states that extracts from a plant called Petasites hybridus were effective in reducing migraines, in recurrent migraine sufferers. That plant extract contains two compounds, called petasine and isopetasine, both of which are known to be leukotriene biosynthesis inhibitors. Accordingly, that report appears to confirm the findings and assertions of the Inventors herein, that drugs which reduce activity at LTD4 receptors (regardless of the specific mechanism of reduction) can indeed help prevent and reduce migraine attacks, in at least some patients.

Leukotriene Receptor Blockers

In the 1980's and 1990's, various drugs were developed which suppress the effects of leukotriene molecules in blood by binding to and occupying leukotriene receptors on the surfaces of various cells, rather than by suppressing the synthesis of leukotrienes. In general, these types of receptor blockers occupy leukotriene receptors on a “competitive” basis, thereby rendering the receptors unable to bind with naturally occurring leukotriene molecules; however, receptor “antagonists” do not trigger the same type or level of cellular response which is triggered by natural leukotriene molecules.

Receptor blockers are often referred to as “receptor antagonists”, or simply as “antagonists” for convenience. However, the term “antagonist” used in pharmacology includes any compound which opposes and reduces the effects of some other compound. Therefore, the term “leukotriene antagonist” includes both leukotriene receptor blockers, and leukotriene biosynthesis inhibitors.

Accordingly, the more specific term, “leukotriene receptor blocker” is used herein to refer to compounds which suppress the effects of leukotriene molecules by means of an action involving binding to a leukotriene receptor, rather than inhibiting an enzyme involved in synthesizing leukotrienes.

The unwanted side effects caused by the leukotriene D4 receptor blockers which were selected for commercialization apparently are quite low, and at least two drugs in that category have become highly successful and widely-used chronic treatments for asthma, since they can help suppress the bronchial and alveolar constrictions that cause or aggravate asthma attacks. One of these drugs is zafirlukast, is sold under the trademark ACCOLATE by Astra-Zeneca Pharmaceuticals (Wilmington, Del.); it is also referred to in various articles as ICI 204,219 (e.g., Taylor et al 1991, Dahlen et al 1991). Zafirlukast is one of a large number of chemical analogs listed in U.S. Pat. No. 4,859,692 (Bernstein et al 1989).

The other widely used LTD4 receptor blocker is montelukast, sold under the trademark SINGULAIR by Merck and Company (West Point, Pa.). Montelukast is one of numerous analogs listed in U.S. Pat. No. 5,565,473 (Belley et al 1996).

Montelukast and zafirlukast are both known to block LTD4 receptors. Montelukast is described in the Merck Index (12th edition, 1996) as a “selective leukotriene D4 receptor antagonist”, while zafirlukast is described in that same reference as a “leukotriene D4 receptor antagonist”.

As used herein, the phrases “LTD4 receptor blocker” (or antagonist) or “leukotriene D4 receptor blocker” (or simply “D4 blocker” or “D4 antagonist”) all refer interchangeably to a drug that can reduce the activity of the LTD4 class of leukotriene molecules, by means of binding reactions involving leukotriene receptors. However, this does not require that any such blocker must be highly selective, and must act only at receptors that interact with LTD4. For example, montelukast and zafirlukast each reportedly have some level of activity at both LTD4 receptors, and LTE4 receptors (Aharony 1998).

Montelukast and zafirlukast are very complex molecules, and are structurally similar to each other, as can be seen by comparing the molecular structures illustrated in reference works such as the Merck Index. Each of these drugs apparently was developed by taking a portion of a leukotriene molecule and bonding it to a different type of molecule (Bernstein 1998), thereby creating a chimeric molecule that binds to LTD4 receptors, but without triggering the cellular response normally induced by natural LTD4 molecules.

Various other LT receptor blockers have also been reported, including ONO-1078 (Taniguchi et al 1993), LY293111 (Evans et al 1996), BAYx7195 (Boulet et al 1997), pranlukast (Hamilton et al 1998), and various N-carbamoyl analogs of zafirlukast (Brown et al 1998). All of these LT receptor blockers are believed to help control and suppress asthma attacks by competitively binding to and occupying one or more types of leukotriene receptors on bronchial cells and various types of blood cells. The effects of these drugs on asthma sufferers are discussed in various articles such as Busse 1996 (a review article) and other articles cited therein, and in a number of articles published after that review, such as Evans et al 1996, Roquet et al 1997, Boulet et al 1997, Dahlen et al 1997, and Hamilton et al 1997 and 1998.

Montelukast and zafirlukast are both sold in pill form, and can be taken every day for long periods of time. Rather than creating tolerance or dependency problems, these drugs appear to help suppress and reduce ongoing asthma problems, when taken chronically, by helping suppress the hypersensitive immune or allergic responses that often grow cumulatively worse in people who suffer from unwanted and excessive activity of the allergic or other immune systems.

As noted above, neither montelukast nor zafirlukast (nor any other leukotriene receptor blockers) were ever used for, or even tested for, their ability to treat or prevent migraine or cluster headaches, prior to the invention of that new method of treatment by the Applicants herein.

The reason for the apparently complete failure of any drug companies to even test any of their LTD4 antagonist drugs against headaches becomes apparent when the results of the first clinical trials involving those drugs are analyzed. As noted above, montelukast and zafirlukast were both tested in asthma patients as the first, top-priority clinical trials. That line of investigation was indeed the most obvious and promising potential use for those new drugs, based on the clear-cut knowledge that leukotrienes are extremely potent bronchoconstrictors. However, during both of those sets of clinical trials (montelukast and zafirlukast) for treating asthma, headaches were reported as one of the most common and frequent side effects. During clinical trials of montelukast, nearly one-fifth (18.4%) of the nearly 2000 asthma patients who were tested reported that they suffered from headaches when they took montelukast; these data are reported in the packaging insert currently sold with montelukast, and in the Physicians Desk Reference. Similarly, during clinical trials of zafirlukast, 12.9% of the more than 4000 asthma patients who were tested reported that they suffered from headaches when they took zafirlukast; these data are also reported in the packaging inserts sold with zafirlukast, and in the Physicians Desk Reference. In both cases, the percentage of patients who complained of headaches were higher than the percentage of control patients who complained of headaches after taking a placebo.

Clearly, those results teach directly away from the subject invention. In the face of results from large-scale human clinical trials, which clearly showed that headaches were a common complaint of asthma patients who took either zafirlukast or montelukast, and which also showed that headache complaints were higher in treated populations than in placebo-treated control populations, it is not surprising that the companies which owned montelukast and zafirlukast did not suspect that either drug might actually be a preventive agent which could prevent people from getting recurrent headaches.

Accordingly, to the best of the Applicants' knowledge and belief, none of the leukotriene antagonist drugs which are commercially available (including zafirlukast and montelukast) were ever used or even tested for preventing or treating migraine or cluster headaches, prior to their discovery and disclosure.

Due to the lack of any adequate and effective level of success among previously known attempted treatments for migraine or cluster headaches, there remains a pressing, widespread, and severe need for a treatment that can be used, on a chronic and long-term basis, to prevent migraine and/or cluster headaches from occurring, and to reduce their severity when they do occur. This is a serious and important medical need which has been acutely known for decades, by the millions of people who suffer from the excruciating pain of migraine and cluster headaches, and by the tens of thousands of physicians and researchers who have been looking, for decades, for such a treatment, without success.

One object of this invention is to disclose and provide a method for long-term and chronic yet safe administration of a drug which can prevent migraine or cluster headaches (i.e., which can reduce their frequency in a patient who suffers from such headaches).

Another object of this invention is to disclose and provide a method for long-term and chronic administration of a drug that can reduce the duration and/or severity of migraine or cluster headaches, when they do arise.

Another object of this invention is to disclose and provide a method for treating migraine headaches among patients who do not adequately respond to “triptan” drugs such as sumatriptan, naratriptan, zolmitriptan, and rizatriptan.

Another object of this invention is to disclose and provide a method for preventing and treating migraine headaches among patients who are highly susceptible to “triggering” factors (such as perfume, or certain foods), to reduce their susceptibility to acute headaches when they occasionally encounter their triggering factors.

Yet another object of this invention is to disclose and provide a method for coadministering two different types of drugs, which work by completely different and independent mechanisms, to provide better pain relief for migraine and cluster headaches than either drug can provide by itself.

These and other objects of the invention will become more apparent through the following summary and description of the preferred embodiments.

### SUMMARY OF THE INVENTION

Receptor blocker (antagonist) drugs which can suppress the binding of the D4 class of leukotriene molecules to leukotriene receptors can reduce the frequency and severity of certain types of severe headaches referred to herein as “recurrent primary headaches”, including migraine and cluster headaches. Various LTD4 receptor blockers are commercially available, including montelukast and zafirlukast.

In the past, these drugs have been used to treat asthma. However, during the human clinical trials in which montelukast and zafirlukast were tested on asthma patients, headaches were one of the most commonly reported side effects of each of those two drugs. Accordingly, they apparently were never subsequently tested as potential preventive agents, to prevent or reduce migraine or cluster headaches. Despite that reported side effect in asthma patients, montelukast and zafirlukast both have been shown to be generally safe, well-tolerated, and free of serious side effects, even when used every day over a continuous period of years.

It has been discovered by the Applicants that, in at least some patients who suffer from acute recurrent migraine headaches, LTD4 receptor blockers can greatly reduce the frequency, duration, and/or severity of such headaches, even among patients who did not respond adequately to any previously known medical treatment. Accordingly, LTD4 blocker drugs appear to provide an important and highly useful medical treatment to reduce the frequency, duration, and/or severity of migraine headaches, cluster headaches, and possibly other types of recurrent primary headaches, using dosage forms such as pills that can be taken every day without causing tolerance, dependence, or other adverse effects.

Results obtained to date also indicate that LTD4 receptor blockers can also be used in acute treatments for migraine or cluster headaches that have already emerged, or which are showing onset symptoms. In treating acute or onset headaches, an LTD4 blocker drug can be used (i) in combination with a “triptan” drug such as sumatriptan, naratriptan, zolmitriptan, or rizatriptan; (ii) in patients who do not respond adequately to triptan drugs and who need alternate treatment; and/or, (iii) as substitutes or alternatives for triptan drugs, to avoid their overuse among patients who suffer from frequent acute headaches.

### BRIEF DESCRIPTION OF THE DRAWINGS

FIG. 1 is a graph depicting a major drop in the reported frequency of “severe” migraine headaches in an open-label clinical trial of montelukast among long-term migraine sufferers. 53% of the treated patients showed a greater than 50% reduction (random probability less than 0.025) in the frequency of severe attacks, with 41% of the treated patients showing a greater than 60% reduction of severe migraine headaches.

FIG. 2 is a bar graph depicting changes in the mean number of migraine headaches per month, divided into “severe”, “moderate”, and “mild” categories. There was a large decrease in the reported “severe” headaches, and a smaller decrease in the frequency of “moderate” attacks. The small increase in the number of “mild” attacks indicated that daily montelukast treatment helped reduce the intensity of migraine headaches from “severe” to “mild” levels.

### DESCRIPTION OF THE PREFERRED EMBODIMENTS

This invention relates to the use of drugs known as “leukotriene D4 receptor blockers” (also referred to as LTD4 blockers) for preventing or treating recurrent primary headaches (which especially includes acute and severe migraine or cluster headaches). In one embodiment, this invention discloses a method of treating a patient who suffers from such recurrent primary headaches, comprising periodic administration (such as daily ingestion) of at least one LTD4 receptor blocker drug, using a chronic dosage regimen which is therapeutically effective in reducing at least one aspect of recurrent primary headaches in such patient.

In an alternate preferred embodiment, which does not depend on or require an analysis of the particular medical status or response of any specific individual patients, this invention discloses a method of preventive treatment to mitigate recurrent primary headaches, among a population of patients who suffer from such headaches. The preventive treatment comprises periodic administration of at least one LTD4 receptor blocker drug, at a chronic dosage regimen which has been statistically shown, in human clinical trials, to be effective in reducing the frequency, duration, and/or severity of recurrent primary headaches.

In other alternate preferred embodiments, this invention discloses methods of treating acute headaches, either during their onset (approach) phases, or after they have become full-blown, acute, severe headaches, comprising the step of administering at least one LTD4 receptor blocker drug to a patient who is either experiencing the onset symptoms of an acute headache, or suffering from an acute headache that has already emerged. In either of these forms of treatment, the LTD4 receptor blocker can be co-administered with a triptan drug, or with any other prescribed analgesic.

Various LTD4 receptor blocker drugs are known; as noted above, the two best-known such drugs are zafirlukast, (sold under the trademark ACCOLATE) and montelukast (sold under the trademark SINGULAIR). Although both are widely used for treating asthma patients, to the best of the Applicant's belief, neither of these drugs was ever previously used, or even tested, as a potential preventive or treatment agent to help control the recurrent headaches that torment and often incapacitate patients who suffer from severe and acute migraine or cluster headaches. Apparently, the complete lack of interest in this type of use against severe headaches arose from the fact that headaches were the most common unwanted side effects that were complained of by asthma patients, who were tested using these drugs in early clinical trials.

Other LT receptor blocker drugs have also been reported in the literature (citations are provided above), including pranlukast, BAYx7195, LY293111, ONO-1078, and various analogs of zafirlukast as reported in Brown et al 1998. Any of these compounds can be tested for use as disclosed herein to treat recurrent primary headaches, using no more than routine experimentation.

In addition to claiming certain specific drugs in an exemplary list (i.e., zafirlukast, montelukast, pranlukast, BAYx7195, LY293111, and ONO-1078), any desired salt or analog of any such drug can also be used, provided that it is both pharmaceutically acceptable, and therapeutically effective in mitigating migraine headaches when taken on a daily basis. Numerous such salts and chemical analogs have already been disclosed in the U.S. patents cited above and in various other published locations.

In addition, other drugs are known which inhibit the biosynthesis of leukotrienes. Such drugs include BAYx1005, MK-886, MK-0591, ZD2138, and zileuton, as well as numerous other drugs disclosed in the U.S. patents cited above which are assigned to Warner Lambert or to Merck Frosst Canada. As mentioned above, the various U.S. patents on those drugs claimed that they could be used to treat or prevent migraines; however, to the best of the Applicants' knowledge and belief, none of those drugs has ever been used or even tested as headache treatments, and those speculative claims were contradicted by reports showing that in clinical trials of zileuton, headaches were one of the most frequent unwanted side effects. Accordingly, since the mechanisms involved in leukotriene biosynthesis inhibition appear to be somewhat promising despite the apparent prior failures, a relatively low dosage of any such leukotriene biosynthesis inhibitor can be tested in combination with an LTD4 receptor blocker drug, to determine whether the combination of both drugs will be more effective than the LTD4 receptor blocker drug alone at preventing or treating migraines without causing severe unwanted side effects.

It should also be noted that many of the leukotriene biosynthesis inhibitors listed above or disclosed in the various patents cited above were initially created by bonding a first portion, derived from a leukotriene molecule, to a second portion derived from an entirely different type of molecule, thereby creating a molecular chimera. Such chimeras are deliberately designed to bind and cling to (and competitively occupy) proteins which normally react with natural leukotrienes (catalytic enzymes, in the case of LT biosynthesis inhibitors; cell receptors, in the case of LT receptor blockers).

Accordingly, it is likely that at least some of the LT biosynthesis inhibitors disclosed in the above-cited patents are likely to also bind to LTD4 receptors. Such competitive binding can be evaluated easily, using simple in vitro binding assays. Accordingly, any such compound which can function as a bi-functional agent, to both inhibit LT biosynthesis and bind to D4 leukotriene receptors, can be evaluated as disclosed herein, to determine whether it can serve as an effective preventive treatment for migraine and/or cluster headaches.

Analogs with Higher BBB Permeability

The discovery that montelukast can affect at least some types of activities that occur within the brain does not provide definitive proof that montelukast penetrates the blood-brain barrier in substantial quantities, or that an LTD4 receptor blocking drug needs to penetrate the blood-brain barrier in order to prevent migraines, in patients who respond to such treatment. In particular, various blood vessels and other tissues and cells outside the BBB (including blood vessels that supply the dura mater and similar tissues that surround the brain, as well as mast cells (which contain leukotrienes) in and around various blood vessels inside the skull but outside the BBB, are suspected of playing potentially important contributory roles in migraines. It may turn out that LTD4 receptor blockers can prevent migraines by, in effect, breaking or interrupting those (or similar) links in a chain of causative factors.

Nevertheless, in view of the data gathered to date, and particularly in view of the profound and dramatic responses by numerous patients (including a substantial number of patients who have moved from severely debilitated status to effectively cured, headache-free status), it is strongly suspected by the Inventors herein that at least part of the powerful effect montelukast has had on at least some patients is very likely due to a substantial quantity of the drug permeating through the BBB and contacting neurons, glial cells, or other types of cells located inside BBB-protected tissue.

This raises the important likelihood that even better and more effective treatments can be created for various CNS-related disorders, by developing LTD4 antagonist drugs that can permeate through the BBB more readily (i.e., more rapidly and/or in higher concentrations, after oral ingestion of a fixed dosage).

Accordingly, now that a highly valuable use which centers on central nervous system (CNS) functioning has been discovered and disclosed for LTD4 antagonists, LTD4 antagonist drugs can be evaluated in much greater detail for their ability to readily penetrate mammalian blood-brain barriers. Candidate drugs which display that particular trait can be tested for use against primary recurrent headaches, or for various other syndromes and complaints that involve brain and/or spinal cord functioning, and analogs of commercially-available LTD4 antagonist drugs (such as montelukast and zafirlukast) which have higher levels of BBB permeability can be evaluated for use in preventing recurrent primary headaches

Leukotriene antagonist compounds having higher levels of BBB permeability than the commercially available asthma treatments can also be identified and evaluated for use in treating other CNS disorders as well. As discussed in provisional patent application serial No. 60/126,627, filed on Mar. 29, 1999 by the same Applicant herein (Sheftell), the discovery that leukotrienes are involved in acute headaches which can be prevented using leukotriene receptor blocker drugs also suggests that, in at least some patients, leukotrienes may be involved as causative or aggravating agents in other types of CNS disorders, possibly including progressive neurodegenerative diseases such as Parkinson's disease, Alzheimer's disease, and Huntington's chorea. Accordingly, the initial discovery and disclosure by the Applicants herein, involving acute headaches, is likely to lead to additional developments in the use of LTD4 antagonist drugs (and possibly LT biosynthesis inhibitors) for treating a variety of CNS disorders. Clearly, any such use will depend on the ability of any such candidate drug to permeate through the BBB and contact neurons and/or glial cells inside the CNS.

The physiology and functioning of the BBB is discussed in numerous reference works; recent monographs and review articles include Brightman et al 1992, Rubin 1999, Pardridge 1999, Kniesel et al 2000. Various methods and models used for animal testing of BBB permeability are reviewed in Bonate 1995, and some early efforts to create in vitro (cell culture) models of evaluating BBB permeability are discussed in Townsend et al 1995 and DeBoer et al 1999.

Other articles focus more specifically on various methods of getting candidate drugs through the BBB and into brain tissue. Most such methods generally fall into one of three categories:

(1) manipulating the BBB to render it more permeable for a brief period of time, such as by intravenous injection of an osmotic-manipulating solution shortly before an anti-cancer drug is injected into a patient with brain cancer;

(2) binding “passenger” compounds to antibodies that can bind to and exploit a BBB transport system such as the transferrin system; and,

(3) modifying candidate drug compounds, to increase their lipophilicity or otherwise increase their ability to penetrate the BBB.

All of these methods are discussed in detail in various review articles, including Langer 1989, Abbott 1996, Begley 1996, Pardridge 1998, Kroll et al 1998, and Rochat et al 1999, and in numerous other research reports, cited in these review articles.

Clearly, the third approach listed above holds the most promise, for administering leukotriene antagonist drugs that will be taken on a daily or other chronic basis, in a manner comparable to the daily oral ingestion of montelukast or zafirlukast tablets or capsules to reduce the frequency and severity of migraine headaches.

One approach to modifying drug compounds in a manner that can increase their BBB permeability deserves note. It is a well-known principle that drugs which are relatively lipophilic tend to cross mammalian blood-brain barriers more readily and in higher concentrations. The term “lipophilic” (often used interchangeably with “oleophilic”) indicates that a compound has an affinity for non-polar, oily/fatty substances or surfaces.

Most lipophilic compounds tend to be relatively hydrophobic as well; this implies that if such compounds are suspended in an aqueous mixture, they will tend to cling to non-polar molecules or surfaces, in a manner which minimizes their area of surface contact with water. However, since some compounds (including various alcohols, detergents, etc.) have relatively high levels of solubility in both water and oily solvents, the terms “lipophilic” and “hydrophobic” are not entirely equivalent or interchangeable.

Because lipophilic drugs tend to permeate through the blood-brain barrier more readily than hydrophilic drugs, it is often possible to modify a known compound (which can be regarded as the initial, starting, or “referent” compound) in a manner that increases its BBB permeability, by modifying the known/starting compound in a manner that slightly increases its lipophilicity. This can be done by various methods known to those skilled in organic chemistry, such as by modifying pendant groups that are bonded to the central structure of a molecule. As examples, a lower alkyl group might be added as a pendant group to a referent compound; a slightly longer alkyl group might be substituted for one that is already present; or an atom or group with a lower degree of polarity might be substituted for a highly polar atom or group. Any of these substitutions (or various others, as discussed in articles such as Kroll et al 1998, Rochat et al 1999, etc., cited above) can create analogs that are more lipophilic, and more readily able to penetrate blood-brain barriers, than the starting/referent compound.

The lipophilicity of candidate compounds often can be predicted with fairly good accuracy by a skilled chemist, just by considering the constituents of the molecule, especially if the molecule is a related analog of another compound with a known level of lipophilicity. Regardless of the accuracy of such a prediction, the lipophilicity of any candidate drug molecule can be measured using simple tests, such as measuring how much of the compound goes into the organic phase, and how much goes into the water phase, when a fixed amount of the compound is stirred into a container that contains both water and a non-polar organic solvent.

Accordingly, analogs and derivatives of montelukast, zafirlukast, or any other known or hereafter-discovered leukotriene antagonist drug can be created which are more lipophilic than existing commercially-available leukotriene antagonist drugs. Such analogs and derivatives offer good candidates for evaluation in animal tests, to identify LTD-4 antagonist compounds that penetrate the BBB in relatively high concentrations, using methods such as described in Bonate 1885. Compounds that show an enhanced ability to permeate mammalian BBB's in one or more animal models will offer preferred candidates for subsequent testing to evaluate safety and side effects in animals. Compounds which perform best in animal tests can be tested for safety and efficacy in human clinical trials.

As noted above, a number of pharmaceutical companies (including Merck, Astra-Zeneca, Warner-Lambert, and Abbott) have already created and patented thousands of analogs of their commercialized LTD4 receptor blocker drugs and LT biosynthesis inhibitors. Those compounds are listed in numerous U.S. patents, such as U.S. Pat. No. 5,565,473 (Belley et al 1996).

For various reasons, based on the results of various screening tests, those other compounds were not chosen for commercialization, to treat asthma patients. However, it must be recognized that, during the animal and clinical tests that focused on identifying the best candidate drugs for treating asthma, a bronchial and pulmonary disorder, the ability of those candidate drugs to permeate through the BBB was of little or no interest.

By contrast, now that a potentially important and valuable use for leukotriene antagonist drugs involving an important CNS activity has been disclosed by the Applicants, the ability of candidate drugs to permeate through the BBB becomes of substantial and even great interest. Accordingly, the hundreds or even thousands of already-known LTD4 antagonist compounds that have already been identified and patented by the pharmaceutical companies listed above (and possibly others as well) can be re-evaluated, to measure the ability of any of those drugs to permeate through the BBB. Those which are likely to have higher levels of BBB permeability than their commercialized cousins (based on appraisals of their chemical structures by skilled organic chemists) can be re-synthesized, or taken out of storage, and tested for actual BBB permeation rates. Candidate compounds which show higher levels of BBB permeation can then become “lead” candidates for further evaluation, in tests which measure the ability of such candidate compounds to treat CNS disorders.

Overview of Clinical Trial

Initial results which were gathered in an “open-label” trial involving 17 patients, as described in more detail below, strongly indicate that LTD4 blocker drugs are effective in reducing the frequency, duration, and/or severity of migraine headaches, among at least some patients who are susceptible to such headaches. All 17 patients completed the study, which consisted of a 2-month period to establish baseline values (including frequency and severity of migraine headaches), followed by a 3 month treatment phase using montelukast. The treatment was quite well tolerated; no adverse events were reported by any of the patients.

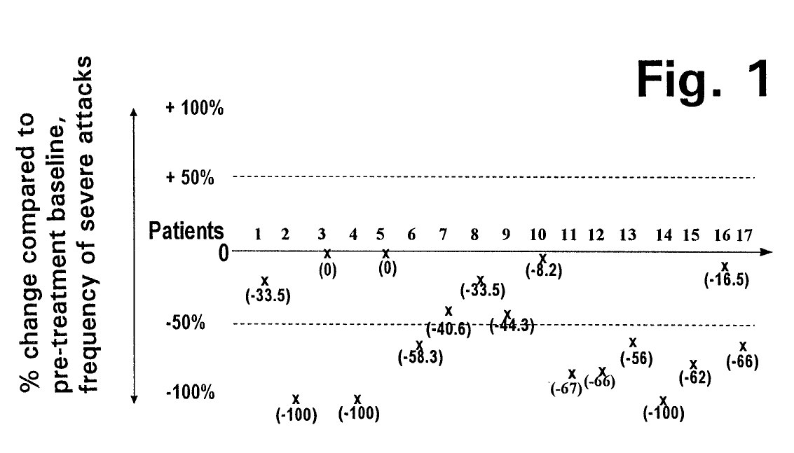

Fifty-three percent of patients who suffered from severe migraine attacks showed a greater than 50% reduction (probability less than <0.025 percent) in the frequency of severe attacks, with 41% of such patients showing a greater than 60% reduction in severe attacks. These results are shown in FIG. 1.

Because of the way the data were processed and analyzed, relatively small increases were seen in occurrence rates for “moderate” or “mild” migraine headaches. These increases represent a substantial decrease in the severity of reported headaches, many of which moved out of the “severe” category and into the “moderate” category (as well as out of the original “moderate” category and into the “mild” category). All patients who responded to the drug rated the drug as excellent.

This Applicants do not claim or assert that all patients who suffer from migraine or cluster headaches will benefit from treatment using an LTD4 receptor blocker. Nevertheless, this invention discloses that at least some such patients will benefit substantially from daily or other chronic administration of an LTD4 receptor blocker drug. Such treatment can reduce, in at least some patients, at least one and usually two (or even all three) of the three main quantifiable traits of recurrent severe headaches (i.e., the frequency, duration, and/or severity of such headaches).

In one preferred embodiment, an LTD4 blocker drug is administered in oral form. A “unit dosage” form with a predetermined quantity of the drug (such as a tablet, capsule, or other pill) is generally preferred, but other oral forms (such as syrups) can be used if desired. The drug is administered at a suitable dosage rate (such as one or two pills per day) on a chronic and long-term basis (such as for months at a time). When administered in such manner, the drug can help prevent and/or reduce the frequency of migraine and/or cluster headaches (and possibly other types of severe recurrent primary headaches as well), and can help reduce the duration and/or severity of such headaches if and when they do occur.

As used herein, terms such as “preventing” and/or “treating” headaches are used broadly. “Preventing” headaches refers to a treatment which reduces the occurrence or frequency of migraine or cluster headaches (which typically can be expressed in terms such as the average number of headaches per month, or per year). “Treating” headaches is a broader term, and includes a drug treatment that can effectively reduce the frequency of such headaches, the duration of such headaches, or the severity of such headaches (which includes a reduction in pain intensity, a reduction in side effects such as nausea, and other such effects that would be regarded as beneficial by a migraine sufferer).

As used herein, any reference to “treating” migraine or cluster headaches using an LTD4 blocker drug also includes the use of such drugs to increase the potency or efficacy of other drugs (such as aspirin, acetaminophen, ibuprofen, naproxen, sumatriptan, ergotamine, or other analgesics) in treating migraine or cluster headaches.

As used herein, all references to “headache” refer to migraine headaches and/or cluster headaches, as those terms are used and interpreted by physicians who specialize in treating such headaches, and as defined in the medical textbooks cited above. It is recognized by the Applicants that treatment using an LTD4 blocker drug may be able to also help reduce the frequency or severity of other types of headaches as well (especially including certain types of severe recurrent headaches such as tension headaches, which for various reasons are not classified as migraine or cluster headaches). However, this current patent application does not cover or claim the use of LTD4 blocker drugs as a general treatment for any and all types of headaches.

Instead, this current application is limited to the use of LTD4 blockers as agents for reducing the frequency, duration, and/or severity of “recurrent primary headaches” (which especially includes migraine and cluster headaches), since there is a major and pressing need for such treatment. Prior to this discovery, there have been no adequate drug treatments that can accomplish those goals in treating either migraine or cluster headaches. A new drug treatment that can achieve those results would be an extraordinary blessing and relief, for the millions of people who suffer from severe and often debilitating migraine and/or cluster headaches.

One of the primary advantages of using an LTD4 blocker drug on a chronic basis to help prevent migraine or cluster headaches is that such drugs apparently do not create any problems of tolerance or dependency. Instead, based on their results in treating asthma, it appears that these drugs are likely to help suppress, control, and reduce, over the long term, various types of gradually cumulative problems which are characteristic of overly-sensitized immunological activity, in which a patient's immune or allergic system keeps getting primed, triggered, or otherwise perturbed in ways which disrupt its desired “stand-by” status, and which generate repeated episodes of unwanted activity, inflammation, and other problems.

If such results continue to be evidenced by chronic daily treatments which last for years without interruption, it appears that LTD4 blocker drugs may offer an ideal approach to a long-term preventive (“prophylactic”) treatment to reduce the number of episodes of acute migraine or cluster headaches, and to reduce the number of “early onset” episodes which indicate the approach of a migraine or cluster headache in ways that require immediate medical intervention, using sumatriptan or other powerful analgesics, to prevent or reduce the onset or severity of a full-blown acute attack.

Short-Term Treatment for Emergent Headaches

It is not asserted herein that LTD4 blocker drugs will perform with a high degree of efficacy, if used by themselves for short-term treatment of a migraine and/or cluster headache that has already commenced, either during the early-onset stage, or after it has become a full-blown acute headache. Nevertheless, such use can be made of LTD4 blocker drugs, if desired, and some patients may respond.

In a generally preferred mode of treatment, an LTD4 blocker preferably should be administered in either or both of the following manners: (i) in a chronic (such as daily) manner, to allow its effects to build up over a period of days or weeks; and/or, (ii) in conjunction with any other type of analgesic (such as aspirin, acetaminophen, ibuprofen, or naproxen) or acute migraine treatment (such as ergotamine or a triptan drug), if and when an acute migraine or cluster headache begins to emerge. It is believed that, by helping reduce and suppress the activity of leukotrienes at LTD4 receptors, such drugs can provide a form of treatment which can act in an additive and possibly synergistic manner, to increase and improve the efficacy, speed, and other beneficial results of other types of analgesic drugs that are used to treat migraine or cluster headaches.

More information on the subject of acute treatment is provided in Example 3, below, including one example of a successful “rescue”, by a woman who was able to abort and prevent an emerging migraine headache by taking a double-dose of montelukast, after failing to take her normal prescribed dosage of montelukast for several days after her prescription ran out.

In addition, it is believed that LTD4 blocker drugs may be able to offer substantial pain relief and other benefits to migraine patients who are not adequately responsive to triptan drugs, ergotamine, or other analgesics (such as aspirin, acetaminophen, ibuprofen, or naproxen). Roughly 30% of patients who are suffering from severe migraine headaches, and who are treated by a specific triptan drug, do not receive adequate relief from that drug. When this occurs, other triptans are usually tested. Although a different triptan drug may help, a residual group of about 10 to 20% of all migraine patients do not respond adequately to any of the known triptan drugs. Patients who are non-responsive to triptan drugs, and patients who have numerous and frequent attacks (such as about 5 per month or more), are especially promising candidates for treatment using LTD4 blockers, either alone or in conjunction with a triptan drug or any other known analgesic.

In addition, as noted in Example 3, below, a number of patients suffering from emerging headaches have reported that a combination of an LTD4 blocker drug and a triptan drug provided them with substantially better pain relief than they were accustomed to receiving from a triptan drug alone.

Although orally ingestible formulations are generally preferred for such use, injectable formulations can also be used, especially for very severe headaches and/or in people suffering from nausea and/or vomiting, who may be unable to keep down an ingested oral formulation. Alternately, rectal suppositories, percutaneous patches, or other modes of administration can also be used by people suffering from nausea and/or vomiting.

It should be noted that the actions of leukotrienes and leukotriene antagonists tend to vary substantially, in sometimes inconsistent ways. For example, in some situations, leukotrienes cause vasoconstriction (i.e., narrowing of blood vessels), as described in Broughton-Smith 1989 and Menger 1994. But in other situations, leukotrienes appear to cause vasodilatation which is the opposite effect. As examples of this apparent inconsistency, the abstract of Ortiz et al 1995 opens with, “Cysteinyl-leukotrienes cause contractions and/or relaxations of human isolated pulmonary vascular preparations”, and closes with, “The mechanical effects of LTD4 on human pulmonary vasculature are complex and are mediated via at least two types of cysteinyl-leukotriene receptors.” Similarly, the abstract of Ford-Hutchinson et al 1986 states, “[leukotrienes] may have important cardiovascular actions through mechanisms involving either vasoconstriction or indirect vasodilatation.”

These apparently inconsistent and paradoxical activities of LT's seem to mirror, in some respects, certain paradoxical aspects of migraine headaches, which typically involve vasoconstriction during the early stages, followed by the opposite activity, vasodilation (which may be an overcompensating response), during the later stages.

Dosages and Modes of Administration

The preferred dosages for any LTD4 receptor blocker selected for use as disclosed herein will depend on various factors, including the age and body weight of a patient taking the medication, etc. In general, one of the primary initial goals of such drug therapy is to establish a daily oral dosage, so that a single convenient “unit dosage” formulation (usually a pill, such as a tablet, capsule, etc.) can be taken by a patient each day. The dosage levels that have already been established for the anti-asthma formulations of zafirlukast (“ACCOLATE”, which normally is taken twice a day) and montelukast (“SINGULAIR”, which normally is taken once a day) offer a good starting point for evaluating preferred dosages that will have maximum beneficial effects in preventing migraine headaches. Evaluative tests to optimize the daily dosages for various patients with particular migraine patterns or severities can be carried out using no more than routine experimentation.

It should also be noted that capsules tend to be well suited for providing a plurality of microencapsulated quantities of an LTD4 receptor blocker drug. Different formulations and thicknesses for the microencapsulating material can be used, to provide an array of tiny pellets that will provide a sustained-release formulation, in a single enclosing capsule that can be taken once a day.

If desired, other types of oral formulations (such as syrups or other liquids, lozenges, troches, etc.) can be developed, and may be well-suited for patients who suffer from nausea. Various types of non-oral formulations (including injectable formulations, nasal sprays, rectal suppositories, transdermal patches, etc.) can also be developed; such non-oral formulations may be preferred for acute treatment of migraines or cluster headaches that have already commenced, especially for patients who suffer from nausea during such headaches.

As another optional approach, an LTD4 blocker can be incorporated into a single tablet, capsule, or other formulation with one or more other drugs, to provide additive or synergistic treatment of migraines, either on a chronic preventive basis, or on an acute treatment basis.

Similarly, two or more LT antagonist drugs can be provided in a single formulation, if desired. For example, an LTD4 receptor blocker can be used along with a leukotriene biosynthesis inhibitor and/or a second LT receptor blocker which blocks a different type of LT receptor.

It should be noted that the currently available LTD4 receptor blockers, zafirlukast (“ACCOLATE”) and montelukast (“SINGULAIR”), typically require about 4 weeks (and sometimes more) of daily oral ingestion of tablets before noticeable effects are seen in reducing the frequency of asthma attacks. However, the articles cited above, reporting the testing of various LT antagonist drugs on asthma patients, indicate that such agents exert a variety of physiological effects within 24 hours of administration. It also should be recognized that after this new use (i.e., using LT receptor blockers to prevent or treat migraine and cluster headaches) is recognized and evaluated by the medical community, it is likely that formulations (including IV or intramuscular injectable formulations, nasal sprays, etc.) can be developed which will not suffer from a prolonged lag time before they become effective in reducing the frequency of migraine headaches. Such non-oral formulations can be used for any desired period of time; for example, they can be used as initial treating agents, to quickly establish a desired level of the drug in circulating blood. Subsequent use of more convenient oral tablets or capsules can be used thereafter, to sustain the desired levels of a LTD4 receptor blocker drug in the blood.

Labelled Packages in Combination with LTD4 Blockers

This invention also discloses an article of manufacture, comprising a LTD4 receptor blocker inside a labelled package which encloses and protects the drug, wherein the labelled package indicates to physicians and purchasers that the D4 blocker drug inside the package is effective, if taken on a daily or other chronic basis, in reducing migraine headaches (or, alternately or additionally, one or more other types of recurrent primary headache), in at least some patients who suffer from such headaches.

This type of article of manufacture, where the label is an essential element of the claimed item, and wherein the label cannot be separated, excised, or divorced from the drug contained inside the package, reflects the fact that under the laws which apply to drugs sold for human use, the drug and its labelled package are regarded as a single indivisible and integral item of commerce. It is illegal to sell such drugs, no matter how safe or effective they may be, unless they are packaged and labelled in a manner that has been approved by the Food and Drug Administration, in the United States (or by similar agencies in other countries).

Preferably, the drug inside the labelled package should be orally ingestible, for convenience of use. Even more preferably, the orally ingestible formulation should be a “unit dosage form”, such as a tablet, capsule, or other pill which has a pre-measured quantity of the drug in each pill.

### EXAMPLES

### Example 1

Open-Label Trial

An open-label clinical trial was organized and conducted at The New England Center for Headache (Stamford, Conn.), which specializes in treating severe headaches, including migraine headaches. Seventeen adult patients, all of whom suffered from “migraine without aura” as defined by International Headache Society (IHS) criteria, were selected to participate. Each patient went through a 2-month baseline evaluation, followed by a 3-month trial period using daily ingestion of montelukast (an LTD4 receptor blocker) in tablet form. Fourteen females and 3 males, between the ages of 14 and 64 (mean age 45.5) were inducted into the study. The mean for “years suffering from migraine” was 24.6, with a range of 5 to 54 years. Although most (n=12) had occasional episodes of episodic tension-type headache (ETTHA), any patients with chronic tension-type headache were excluded. Inclusion criteria included diagnosis of IHS migraine without aura, the ability of patients to differentiate migraine from ETTHA, and willingness to keep accurate headache calendars for the two-month baseline period and the three-month treatment phase. Patients with chronic tension-type headache, transformed migraine, analgesic/ergot/triptan overuse, or failure on two or more previous trials on preventive agents were excluded from the study. No changes in concomitant prophylactic regimens (n=11 of the 17 total) were allowed during the baseline (BL) period or during montelukast treatment.

In general, patients were selected only if they had not responded in an adequate and satisfactory manner to other treatments for migraine. Patients were not selected if they had totally failed to respond to any other preventive medications; accordingly, those who were selected generally comprised: (i) “partial responders” who were already receiving some form of preventive treatment when they entered the study, but who were still experiencing a sufficient number of migraine attacks to warrant further steps to improve their treatment; and, (ii) patients who had elected not to be on other preventive medications, because of side effects such as fatigue, weight gain, dry mouth, sexual dysfunction, etc. All patients who were chosen for the study, and who were informed of the drug that would be used in the study, were happy to take montelukast on a daily basis, in view of its very low level of side effects.

Each selected patient went through a 2-month “baseline” period. During that period (and also during the 3-month trial period), no medications, hormones, or other medical treatments were altered. Upon being selected, each patient was required to keep a “headache calendar” for at least two months prior to entering the trial, in order to establish baseline or “control” data which could be compared to the results of the trial period. Daily administration of montelukast, in 10 mg tablets taken once per day, began after the 2-month baseline period had been completed by a patient. Patients who typically awoke with migraine attacks were dosed at bedtime. Patients who reported that their attacks typically occurred during the afternoon or evening were dosed accordingly.

During the baseline period and the study period, the frequency, severity, and duration of migraine attacks were recorded on a calendar or log book, by each study participant. All patients who did not show a decrease of at least 50% in frequency of attacks within 2 months after commencing montelukast treatment at 1 tablet/day had their dosages increased to 2 tablets per day, and were evaluated 4 and 8 weeks later.

In addition, patients who reported specific sensitivity to perfume as a migraine triggering factor were evaluated for reduced sensitivity to perfume triggering.

The trial was conducted as a prospective, “open label” trial. As such, blinding procedures were not used to conceal which drug a patient was taking; instead, each patient was informed of exactly what drug he or she would receive during the trial.

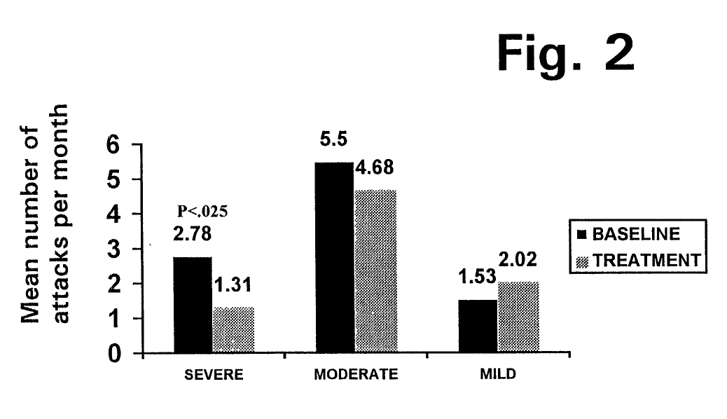

As shown in FIG. 2, the mean number of monthly attacks that were categorized as “severe”, in baseline patients before montelukast treatment began, was 2.78 (range 0 to 4.5). This frequency level was reduced by more than half (p<0.025) by montelukast treatment, to a mean value of 1.31 severe attacks per month. Percentage reductions for individual patients are displayed in FIG. 1; these data indicate that more than half of the patients (53%) patients showed a reduction of more than 50% in the frequency of severe attacks, with 41% of patients showing a greater than 60% reduction in frequency.

Despite the shift of substantial numbers of headaches out of the “severe” category and into the “moderate” category, there was nevertheless a reduction in the total number of reported “moderate” attacks as well, as shown in FIG. 2. The mean number of “moderate” attacks per month reported by patients during the baseline periods was 5.5, with a range of 1 to 9. This mean value decreased to 4.68 among treated patients. Although this numerical difference was not statistically significant on an absolute basis, it was in fact highly significant, since the overall frequency dropped even though several patients reported increases in the number of “moderate” headaches they suffered, as their headaches shifted out of the “severe” category and into the “moderate” category. In addition, even when patients were included whose attacks had decreased from “severe” to “moderate” levels, 70% of all reporting patients reported a decrease in the number of moderate headaches they suffered.

For similar reasons, the number of “mild” headaches that were reported increased slightly, from a pre-treatment baseline level of 1.53 to a treatment level of 2.02. Instead of being a negative result, this reflects the number of patients who reported substantial decreases in the intensity of their headaches, from “severe” or “moderate” levels before treatment, to “mild” levels during treatment.

A total of 4 of the 17 patients were escalated from 10 mg once daily, to 10 mg twice daily, during the study (2 patients shifted after 1 month, and 2 patients shifted after 2 months). All 4 of these patients experienced a reduction in the frequency of “severe” attacks for the remaining 1 or 2 months of their treatment period. Several other patients were eligible for a dosage increase, but they declined, in view of their satisfaction with what appeared to them to be a substantial and highly welcome reduction in the severity of their attacks.

None of the patients reported any adverse effects; in specific, none of the tested patients reported any increase in headache frequency or severity.

Two of the 17 patients (regarded as “non-responders”) reported no change in frequency or severity levels for their migraine attacks.

Of the remaining 15 patients, 13 had a 33% or greater reduction in the frequency of severe attacks. All 13 of those patients rated the treatment as “excellent”, and were highly reluctant to terminate the treatment when their test period ended.

Additional and larger trials (including double-blinded placebo-controlled trials) can be carried out, using any selected type or formulation of an LTD4 blocker, on any type, class, or group of recurrent severe headaches, as necessary to evaluate such formulation and to obtain governmental approval to label that drug formulation for sale and use as a safe and effective treatment for such headaches.

### Example 2

Post-Publication Results

The Applicant herein (Sheftell) initially published his leukotriene-migraine findings in abstract form and as a poster session in mid-1999, at a conference of physicians who specialize in treating headaches. That presentation was summarized shortly thereafter in Family Practice News, a newsletter for physicians, and it was followed in February 2000 by a full article in a refereed journal (Sheftell et al, “Montelukast in the prophylaxis of migraine: “potential role for leukotriene modifiers,” Headache 40: 158-63 (2000)).

Following publication of the abstract and article, Sheftell has received numerous unsolicited letters and comments from patients as well as physicians, stating that they have used montelukast or zafirlukast for chronic migraines, and have benefitted more from that treatment than from any other treatment they have ever received (or prescribed, in the case of treating physicians).

One example of an unsolicited letter was received from a physician who works in the Midwest. That physician, a family practitioner, stated, “I personally have had migraines for eighteen years. They have been especially bad the past ten years. My neurologist and I have had a great deal of trouble controlling them. However, since starting on [montelukast] ten days ago, I have been headache free—the longest headache free period I have had in ten years. Thank you for doing your study and giving me (and hopefully millions of others) a way to control this affliction.”

Similar commentaries by other patients who have been dramatically helped by this treatment can be found on an Internet website, called “Ronda's Headache Page” (http://www.migrainepage.com), an information and support page for people who suffer from migraines and other acute headaches. One such posting reads: “Now hear this—I'm cured! 10 mg of Singulair (asthma drug/I don't have asthma) in the morning and 10 mg at night (that equals 20; just 10 didn't quite work all the way) has put SEVEN MONTHS OF A CONTINUOUS HEADACHE out of my head! No headache. The usual triggers don't trigger it. You heard me right. If nothing else is working for you, try this. It takes a couple weeks to build up and work. Anyway, I hope somebody is helped by this. And remember, they'll start you on 10 mg/day, so if that doesn't work, try 20 mg/day (you have to wait another two weeks for the right build up). Okay?”

Obviously, not all patients respond in such a clear-cut manner; nevertheless, the ability of this treatment to make life-changing differences in some patients has been shown clearly and repeatedly.

### Example 3

Treatment of Emergent Headaches

On several occasions, the Inventors have been informed, by patients with long histories of triptan use for recurrent migraines, that treatment using both montelukast and a triptan drug, for an emerging or full-blown headache, provided them with highly positive results, which were substantially more effective than the results those patients were accustomed to receiving from triptan drugs only.

In addition, on one occasion, a patient (female, in her 40's) who had been taking montelukast for several months as a migraine preventive treatment ran out of pills, and did not get a refill immediately. On about the third day of not taking any montelukast, she began to sense that a migraine headache was emerging. She immediately called her doctor and obtained a refill. On her own volition, she took two capsules, instead of one. She reported that the emerging headcache was completely aborted and prevented.

Thus, there has been shown and described a new and useful method for treating patients who suffer from recurrent severe headaches, including migraine headaches and cluster headaches. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

References

Abbott, N. J., et al, “Transporting therapeutics across the blood-brain barrier,” Mol Med Today 2: 106-13 (1996)

Aharony, D., “Pharmacology of leukotriene receptor antagonists,” Am J Respir Crit Care Med. 157: S214-8 (1998)

Begley, D. J., “The blood-brain barrier: principles for targeting peptides and drugs to the central nervous system,” J Pharm Pharmacol 48: 136-46 (1996)

Bernstein, P. R., “Chemistry and structure-activity relationships of leukotriene receptor antagonists,” Am J Respir Crit Care Med 157: S220-5 (1998)

Bonate, P. L., “Animal models for studying transport across the blood-brain barrier,” J Neurosci Methods 56: 1-15 (1995)

Boulet, L. P., et al, “Inhibitory effects of BAYx7195, a CYS leukotriene 1 receptor antagonist, on allergen-induced asthmatic responses,” Ann Allergy Asthma Immonol 79(2): 155-61 (1997)

Bray, M. A., et al, “Leukotriene B4: a mediator of vascular permeability,” Brit J Pharmacol 72: 483-486 (1981)

Bray, M. A., “The pharmacology and pathophysiology of leukotriene B4,” Brit Med Bull 39: 249-254 (1983)

Brown, M. F., et al, “N-carbamoyl analogs of zafirlukast: Potent receptor antagonists of leukotriene D4,” Bioorg Med Chem Lett. 8: 2451-6 (1998)

Busse, W. W. “The role of leukotrienes in asthma and allergic rhinitis,” Clin Exp Allergy 26(8): 868-79 (1996)

Dahlen, S. E., et al, “Inhibition of allergic bronchoconstriction in asthmatics by the leukotriene-antagonist ICI-204,219,” Adv Prostaglandin Thromboxane Leukotriene Res 21A: 461-4 (1991)

Dahlen, B., et al, “Inhibition of allergen-induced airway obstruction and leukotriene generation in atopic asthmatic subjects by the leukotriene biosynthesis inhibitor BAYx1005,” Thorax 52(4): 342-7 (1997)

DeBoer, A. G., et al, “The transference of results between blood-brain barrier cell culture systems,” Eur J Pharm Sci 8: 1-4 (1999)

Diamant, Z., et al, “The effect of MK-0591, a novel 5-lipoxygenase activating protein inhibitor, on leukotriene biosynthesis and allergen-induced airway responses in asthmatic subjects in vivo,” J Allergy Clin Immunol 95(1 Pt 1): 42-51 (1995)

Evans, D. J., et al, “Effect of a leukotriene B4 receptor antagonist, LY293111, on allergen induced responses in asthma,” Thorax 51(12): 1178-84 (1996)

Ford-Hutchinson, A., et al., “Biological actions of leukotrienes,” Hypertension 8 (Part 2): II44-49 (1986)

Ford-Hutchinson, A. W., et al, “Leukotriene B4 a potent chemokinetic and aggregating substance released from polymorphonuclear leukocytes,” Nature 286: 264-265 (1980)

Friedman, B. S., et al, “Oral leukotriene inhibitor (MK-886) blocks allergen-induced airway responses,” Am Rev Respir Dis 147(4): 839-44 (1993)

Gazzaniga, P. P., et al, “Identification of blood leukotrienes in classical migraine,” Headache 27: 211-5 (1987)

Gimbrone, M. A., et al, “Leukotriene B4 stimulates polymorphonuclear leukocyte adhesion to cultured vascular endothelial cells,” J Clin Invest 74: 1552-1555 (1984)

Grossman, M., et al, “An extract of Petasites hybridus is effective in the prophylaxis of migraine,” Intl J Clin Pharm Therapeutics 38: 430-435 (2000)

Hamilton, A. L, et al, “Pranlukast, a cysteinyl leukotriene receptor antagonist, attenuates allergen-induced early- and late-phase bronchoconstriction and airway hyperresponsiveness in asthmatic subjects,” J Allergy Clin Immunol 102(2): 177-83 (1998)

Hamilton, A. L., et al, “Attenuation of early and late phase allergen-induced bronchoconstriction in asthmatic subjects by a 5-lipoxygenase activating protein antagonist, BAYx 1005,” Thorax 52(4): 348-54 (1997)

Hui, K. P., et al, “Effect of a 5-lipoxygenase inhibitor on leukotriene generation and airway responses after allergen challenge in asthmatic patients,” Thorax 46(3): 184-9 (1991)

Knapp, H. R., “Reduced allergen-induced nasal congestion and leukotriene synthesis with an orally active 5-lipoxygenase inhibitor,” N Engl J Med 323(25): 1745-8 (1990)

Kniesel, U., et al, “Tight junctions of the blood-brain barrier,” Cell Mol Neurobiol 20: 57-76 (2000)

Kroll, R. A., et al, “Outwitting the blood-brain barrier for therapeutic purposes: osmotic opening and other means,” Neurosurgery 42: 1083-99 (1998)

LaMancusa, R., et al, “Blood leukotrienes in headache: Correlation with platelet activity,” Headache 31: 409-14 (1991)

Langer, R., et al, “New directions in CNS drug delivery,” Neurobiol Aging 10: 642-4 (1989)

Levine, J. D., et al, “Leukotriene B4 produces hyperalgesia that is dependent of polymorpho-nuclear leukocytes” Science 225: 743-745 (1984)

Lewis, R. A., et al, “Mediation of local homeostasis and inflammation by leukotrienes and other mast cell dependent compounds,” Nature 293: 103-108 (1981)

Marx, J., “The leukotrienes in allergy and inflammation,” Science 215: 1380-1383 (1982)

Moskowitz, M. A., “Basic mechanisms in vascular headache,” Neurol Clin. 8: 801-15 (1990)

Nasser, S. M., et al, “Effect of the 5-lipoxygenase inhibitor ZD2138 on allergen-induced early and late asthmatic responses,” Thorax 49(8): 743-8 (1994)

Nicosia, S., et al, “Receptors for cysteinyl-leukotrienes in human cells,” Adv Exp Med Biol 447: 165-70 (1999)

Ortiz, J. L., et al., “Leukotriene receptors on human pulmonary vascular endothelium,” Brit J Pharmacol 115: 1382-86 (1995)

Parantainen, J., et al, “Clinical aspects of prostaglandins and leukotrienes in migraine,” Cephalalgia 6 Suppl 4: 95-101 (1986)

Pardridge, W. M., “CNS drug design based on principles of blood-brain barrier transport,” J Neurochem 70: 1781-92 (1998)

Pardridge, W. M., “Blood-brain barrier biology and methodology,” J Neurovirol 5: 556-69 (1999)

Piper, P. J., “Formation and action of leukotrienes,” Physiol Rev 64: 749-761 (1984)

Rochat, B., et al, “Drug disposition and targeting: Transport across the blood-brain barrier,” Pharm Biotechnol 12: 181-200 (1999)

Roquet, A., et al, “Combined antagonism of leukotrienes and histamine produces predominant inhibition of allergen-induced early and late phase airway obstruction in asthmatics,” Am J Respir Crit Care Med 155: 1856-63 (1997)

Rubin, L. L., “The cell biology of the blood-brain barrier,” Annu Rev Neurosci 22: 11-28 (1999)

Smith, M. H. J., et al “Leukotriene B: a potential mediator of inflammation,” J Pharmacol 32: 517-18 (1980)

Taniguchi, Y., et al, “The effect of an oral leukotriene antagonist, ONO-1078, on allergen-induced immediate bronchoconstriction in asthmatic subjects,” J Allergy Clin Immunol 92: 507-12 (1993)

Taylor, I. K., et al, “Effect of cysteinyl-leukotriene receptor antagonist ICI 204.219 on allergen-induced bronchoconstriction and airway hyperreactivity in atopic subjects,” Lancet 337(8743): 690-4 (1991)

Townsend, G. C., et al, “In vitro models of the blood-brain barrier to study bacterial meningitis,” Trends Microbiol 3: 441-5 (1995)

1. A method of treating a patient who suffers recurrent primary headaches, comprising periodic administration of at least one LTD4 receptor blocker drug using a chronic dosage regimen which is therapeutically effective in reducing at least one aspect of recurrent primary headaches in such patient.

2. The method of claim 1 wherein periodic administration of the LTD4 receptor blocker drug causes a reduction in at least one aspect of such headaches selected from the group consisting of:
a. frequency of occurrence of such headaches;
b. duration of such headaches, when they occur; and,
c. perceived or reported severity or intensity of such headaches, when they occur.

3. The method of claim 1, wherein the patient suffers from severe recurrent primary headaches which are classified as migraine headaches.

4. The method of claim 3 the LTD4 receptor blocker drug is administered using daily or twice-daily ingestion of an orally-ingestible unit dosage formulation of the LTD4 receptor blocker drug.

5. The method of claim 3 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating migraine headaches when taken on a daily basis.

6. The method of claim 1, wherein the patient suffers from recurrent severe headaches which are classified as cluster headaches.

7. The method of claim 6 wherein the LTD4 receptor blocker drug is administered using daily or twice-daily ingestion of an orally-ingestible unit dosage formulation of the LTD4 receptor blocker drug.

8. The method of claim 6 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating migraine headaches when taken on a daily basis.

9. A method of preventive treatment to mitigate recurrent primary headaches, comprising periodic administration of at least one LTD4 receptor blocker drug, at a chronic dosage regimen which has been statistically shown to be effective in reducing frequency, duration, and severity of recurrent primary headaches.

10. The method of claim 9 wherein periodic administration of the LTD4 receptor blocker drug causes a reduction in at least one aspect of such headaches selected from the group consisting of:
a. frequency of occurrence of such headaches;
b. duration of such headaches, when they occur; and,
c. perceived or reported severity or intensity of such headaches, when they occur.

11. The method of claim 9, wherein the patient suffers from recurrent severe headaches which are classified as migraine headaches.

12. The method of claim 11 the LTD4 receptor blocker drug is administered using daily or twice-daily ingestion of an orally-ingestible unit dosage formulation of the LTD4 receptor blocker drug.

13. The method of claim 12 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating migraine headaches when taken on a daily basis.

14. The method of claim 9, wherein the patient suffers from recurrent severe headaches which are classified as cluster headaches.

15. The method of claim 14 wherein the LTD4 receptor blocker drug is administered using daily or twice-daily ingestion of an orally-ingestible unit dosage formulation of the LTD4 receptor blocker drug.

16. The method of claim 15 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating cluster headaches when taken on a daily basis.

17. A method for treating an emerging acute headache during its onset phase, comprising the step of administering at least one LTD4 receptor blocker drug to a patient who is experiencing onset symptoms of an acute headache.

18. The method of claim 17 wherein the patient suffers from recurrent migraine headaches.

19. The method of claim 17 wherein the patient suffers from cluster headaches.

20. The method of claim 17 wherein the LTD4 receptor blocker drug is co-administered with a triptan drug.

21. The method of claim 17 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating recurrent primary headaches.

22. A method for treating an acute headache, comprising the step of administering at least one LTD4 receptor blocker drug to a patient who is suffering an acute headache, at a dosage which has been shown to be therapeutically effective in reducing the severity or duration of migraine headaches when administered to patients suffering from acute migraine headaches.

23. The method of claim 22 wherein the patient suffers from recurrent migraine headaches.

24. The method of claim 22 wherein the patient suffers from cluster headaches.

25. The method of claim 22 wherein the LTD4 receptor blocker drug is co-administered with a triptan drug.

26. The method of claim 22 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating migraine headaches when taken on a daily basis.

27. An article of manufacture comprising a LTD4 receptor blocker drug inside a labelled package which encloses and protects the drug, wherein the LTD4 receptor blocker drug is in an orally ingestible formulation which is effective in reducing migraine headaches in at least some patients if taken chronically, and wherein the labelled package indicates to physicians and purchasers that the LTD4 receptor blocker drug enclosed therein is effective in reducing at least one type of recurrent primary headache in at least some patients who suffer from such headaches.

28. The article of manufacture of claim 27, wherein the orally ingestible formulation is a unit dosage form.

29. The method of claim 27 wherein the LTD4 receptor blocker drug is selected from the group consisting of zafirlukast, montelukast, pranlukast, BAYx7195, LY293111, and ONO-1078, and salts and analogs thereof which are pharmaceutically acceptable and therapeutically effective in mitigating migraine headaches when taken on a daily basis.
